(19) United States
(12) Patent Application Publication
(10) Pub. No.: US 2025/0359862 A1
(43) Pub. Date: Nov. 27, 2025

(54) RETRACTOR SYSTEM

(71) Applicant: Nicholas A. Qandah, New Hartford, NY (US)

(72) Inventors: Nicholas A. Qandah, New Hartford, NY (US); Melissa D. Coale, Chester, NJ (US)

(21) Appl. No.: 19/226,136

(22) Filed: Jun. 2, 2025

Related U.S. Application Data
(63) Continuation of application No. 18/146,159, filed on Dec. 23, 2022, now Pat. No. 12,318,082.
Application No. 18/146,159 is a continuation of application No. 15/695,079, filed on Sep. 5, 2017, now Pat. No. 11,534,153.

(51) Int. Cl. A61B 17/02; A61B 17/00; A61M 29/00

(52) U.S. Cl. CPC A61B 17/0293; A61B 17/0206; A61M 29/00; A61B 2017/0046

(57) ABSTRACT

A retractor system is provided that allow improved instrumentation and access during a surgical procedure including for instance a lateral approach minimally invasive spinal fusion procedure. Disclosed systems include a handheld retractor, a ring retractor having an armature and a plurality of pivotable blade assemblies, and optional muscle retractors and stepped dilators cooperatively coupled together to form an exemplary system.

### CROSS-REFERENCE TO RELATED APPLICATIONS

This application claims the benefit under 35 USC § 120 of U.S. Nonprovisional application Ser. No. 18/146,159 filed 23 Dec. 2022, which in turn claims the benefit of U.S. Nonprovisional application Ser. No. 15/695,079 filed 5 Sep. 2017, the contents of each of which are hereby incorporated by reference as if fully recited herein.

### TECHNICAL FIELD

Exemplary embodiments of the present invention relate generally to mechanical devices and their use, and more specifically to devices used in connection with surgical procedures.

### BACKGROUND OF THE INVENTION

In the field of surgery in general and lateral approach spine surgery in particular, it is often beneficial to have retractor instruments which are capable of a multi-stepped approach, with each respective step serving a distinct function in the procedure. To that end, devices applicable for at least a lateral approach minimally invasive lumbar spine fusion are provided. Although the devices of the present invention are described in relation to spine surgery specifically, they are relevant and usable within many other surgical fields where access below large portions of tissue or musculature are helpful to a surgeon.

### BRIEF SUMMARY OF THE INVENTION

According to certain embodiments of the present disclosure, a retractor system includes; a handheld retractor having a blade disposed upon a first end portion thereof, a handle disposed upon a second portion thereof, and a first engagement feature disposed therebetween; ring retractor having a substantially rigid generally planar armature enclosing at least part of an area defining an operative site, a plurality of articulated blades coupled to the armature, and a second engagement feature disposed upon a distal face of the armature; wherein the first engagement feature and second engagement feature are complementary structures sized and shaped for rigidly coupling the ring retractor upon the handheld retractor after the handheld retractor has been positioned by an operator.

According to further embodiments of the present disclosure, the first engagement feature is a post and the second engagement feature is a complementary aperture.

According to further embodiments of the present disclosure, the first engagement feature is the outside profile of a portion of the handheld retractor and the second engagement feature is a complementary recess in the ring retractor wherein the shape of the recess substantially approximates the outside profile of the handheld retractor.

According to further embodiments of the present disclosure, the first engagement feature is a post having an internal thread.

According to certain embodiments of the present disclosure; a handheld retractor includes a blade disposed upon a first end portion thereof, a handle disposed upon a second portion thereof, and a first engagement feature disposed therebetween; ring retractor having a substantially rigid armature enclosing at least part of an area defining an operative site, a plurality of articulated blades coupled to the armature, and a second engagement feature disposed upon a distal face of the armature; wherein the first engagement feature and second engagement feature are complementary structures sized and shaped for rigidly coupling the ring retractor upon the handheld retractor after the handheld retractor has been positioned by an operator wherein the first and second engagement features are configured for the armature to be mountable in a proximal orientation upon the handheld retractor.

According to further embodiments of the present disclosure, the handheld retractor is a Deaver Retractor.

### BRIEF DESCRIPTION OF THE SEVERAL VIEWS OF THE DRAWINGS

In the figures, which are not necessarily drawn to scale, like numerals describe substantially similar components throughout the several views. The drawings illustrate generally, by way of example, but not by way of limitation, various embodiments discussed in the claims of the present document.

FIG. 1 shows a side view of a handheld retractor.

FIG. 2A shows a perspective view of a handheld retractor.

FIG. 2B shows an enlarged perspective view of several coupling features disposed upon a handheld retractor.

FIG. 3 shows a perspective view of a dilator.

FIG. 4 shows a perspective view of a dilator.

FIG. 5A shows a perspective view of a muscle retractor.

FIG. 5B shows an enlarged perspective view of several coupling features of a muscle retractor.

FIG. 6 shows a perspective view of a ring retractor.

FIG. 7 shows a rear view of a ring retractor.

FIG. 8 shows a perspective view of a first step in the assembly of an embodiment of a Retractor System.

FIG. 9 shows a perspective view of a second step in the assembly of an embodiment of a Retractor System.

FIG. 10A shows a perspective view of a third step in the assembly of an embodiment of a Retractor System.

FIG. 10B shows a plan view of the third step in the assembly of an embodiment of a Retractor System.

### DETAILED DESCRIPTION OF THE INVENTION

Various embodiments of the presently disclosed apparatus will now be described in detail with reference to the drawings, wherein like reference numerals identify similar or identical elements. In the drawings and in the description that follows, the term “proximal,” will refer to the end of a device or system that is closest to the operator, while the term “distal” will refer to the end of the device or system that is farthest from the operator. Similar, anatomical terms of reference such as dorsal, lateral, anterior, and sagittal shall have their accepted meanings in the arts.

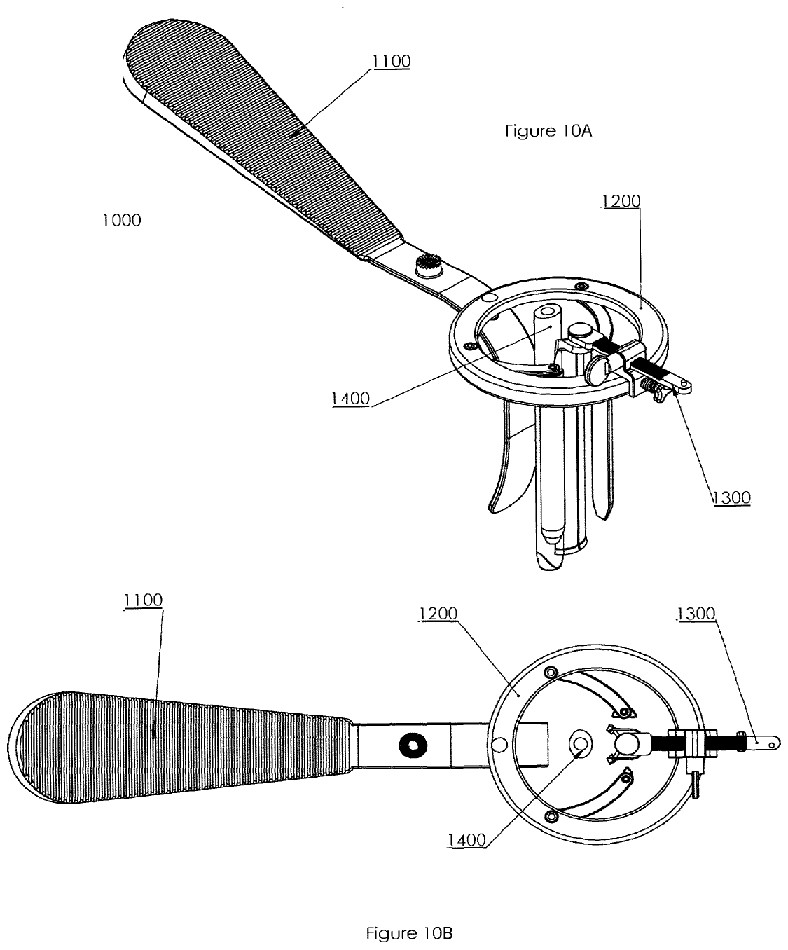

According to a first embodiment of the present disclosure shown in FIGS. 10A and 10B, a Retractor System 1000 comprises a handheld retractor 1100, with the handheld retractor oriented distally from a ring retractor 1200. Additionally, there is a muscle retractor 1300 capable of optionally coupling to the ring retractor, and a set of stepped dilators 1400 which are sized and shaped to be used within the ring retractor either independently or coupled to the ring retractor with an additional armature.

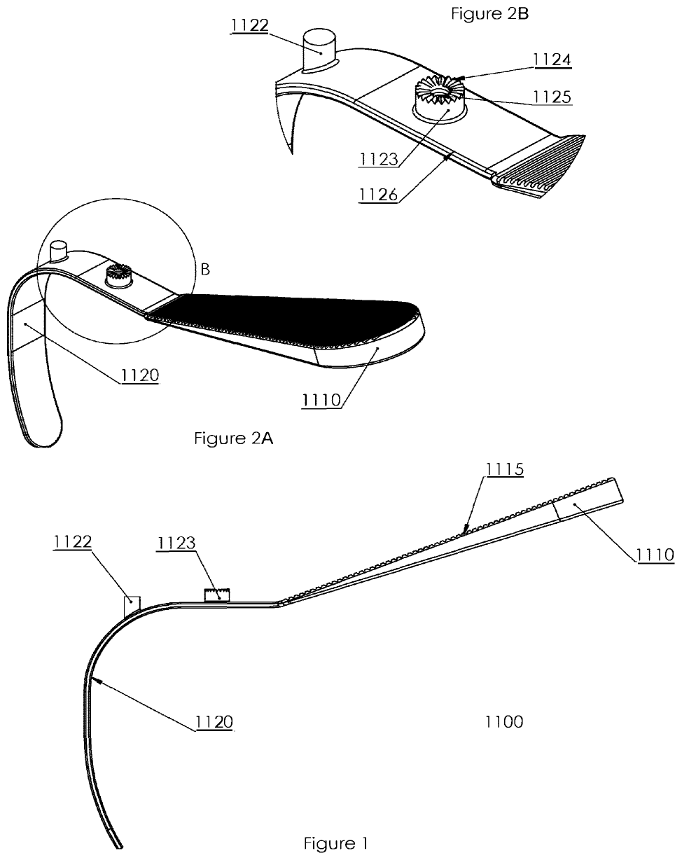

Referring now to FIGS. 1, 2A, and 2B together, an embodiment of a handheld retractor 1100 is shown wherein the retractor has an arcuate blade 1120 composed of a metal or polymer at a first, distal end thereof and an enlarged hand-grip 1110 at a second, proximal end thereof. Blade 1120 is sized and shaped to manually retract large portions of tissue. According to certain embodiments of the present disclosure, the blade 1120 approximates what is referred to as a Deaver Retractor in the surgical arts. The present invention is distinguishable from existing Deaver Retractors at least in that the mid-section of the retractor 1100 has a plurality of features sized and shaped to couple the device to the remainder of the assembly. These are (1) a smooth post 1122 which is a substantially cylindrical body extending proximally from the blade 1120, (2) a ribbed post 1123 which is a hollow body having a smooth exterior, a thread 1125 about its interior, and a plurality of teeth upon its proximal face sized and shaped to be coupled fixedly to a mirror reflection thereof, and (3) a coupling profile 1126 upon a portion of the retractor 1100 between the blade 1120 and handle 1110 which is sized and shaped to fixably receive a portion of ring retractor 1200 thereupon as will be explained later. Handle 1110 is an enlarged portion of the retractor 1100 having a modified surface texture 1115 upon a portion thereof sized and shaped to improve grip of the device when held by the gloved hand of an operator, including for instance a surgeon.

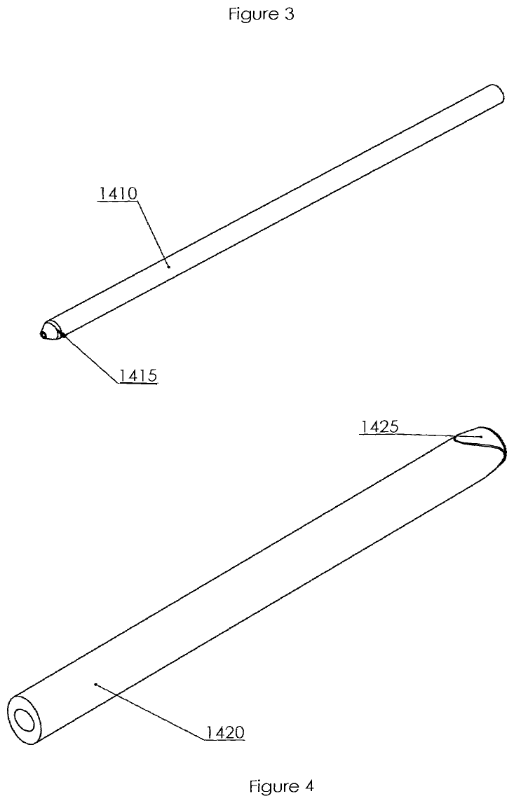

Referring now to FIGS. 3 and 4 together, stepped dilators 1410 and 1420 are shown which when assembled together define dilator 1400. First dilator 1410 is a small tube having a canula extending therethrough along a longitudinal axis of the dilator 1410 with the canula sized and shaped for sliding along a guide wire and a conical tip 1415 near the distal end portion thereof sized and shaped for puncturing tissue. Second dilator 1420 is a larger cross-section tube having an elliptical outer profile and an inner profile sized and shaped to smoothly accommodate the movement of first dilator 1410 therethrough and having a conical tip at the distal end portion thereof with the tip being sized and shaped to puncture and enlarged tissue when pressure is applied thereagainst.

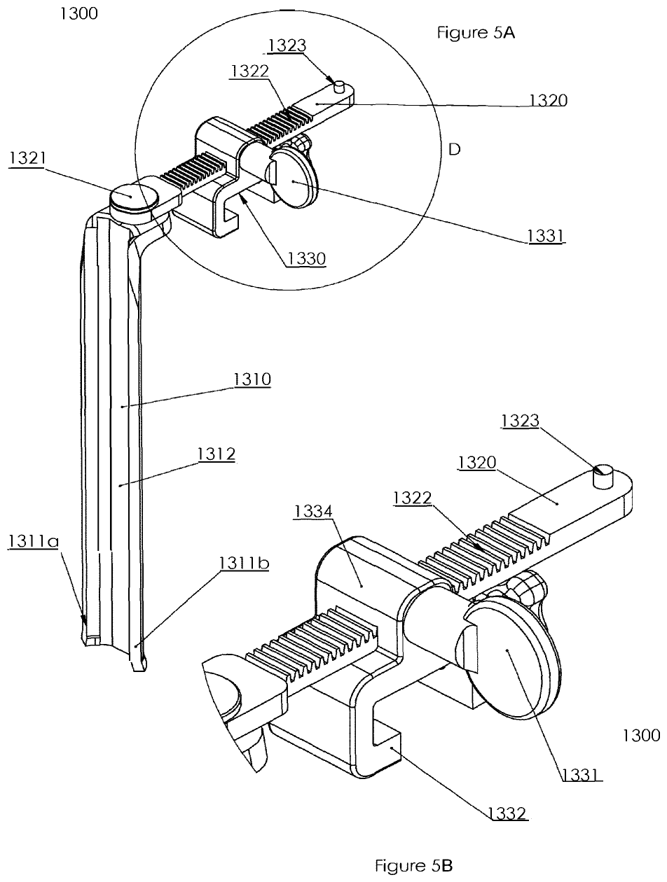

Referring now to FIGS. 5A and 5B together, a muscle retractor 1300 comprises a blade 1310 pivotably coupled to a linear gear 1320, with the linear gear being movably relative to a clamp 1330.

Blade 1310 is an elongated body comprised of a metal or polymer having a curved cross-section with opposing faces 1311a and 1311b turned inwards towards one another and a connecting surface 1312 disposed therebetween. According to certain embodiments of the present disclosure, there is a ridge at junction of faces 1311 (a and b) and surface 1312 thereby defining a rail upon which additional tools or instrumentation may be slideably coupled. Upon the proximal-end portion blade 1310, there is a bend which provides a location for a pivot 1321.

Linear gear 1320 is an elongated body comprised of metal or polymer having a pivot 1321 coupled to the blade 1310 at a distal end thereof, a pin 1323 extending upward from the proximal end portion thereof and a plurality of teeth defining a gear train 1322 disposed therebetween. Clamp 1330 is positionable upon the length of gear train 1322.

Clamp 1330 has a key 1331 extending therethrough with a finger-grasp at a first end thereof and a spur gear at a second end thereof with the spur gear being engaged upon gear train 1322. The second end of the key is enclosed within a body 1334 and sized and shaped to rotate freely therewithin. Clamp 1332 is sized and shaped to engage upon the body of ring retractor 1200, with rotation of key 1331 defining the position of blade 1310 relative to ring retractor 1200 when the device is assembled.

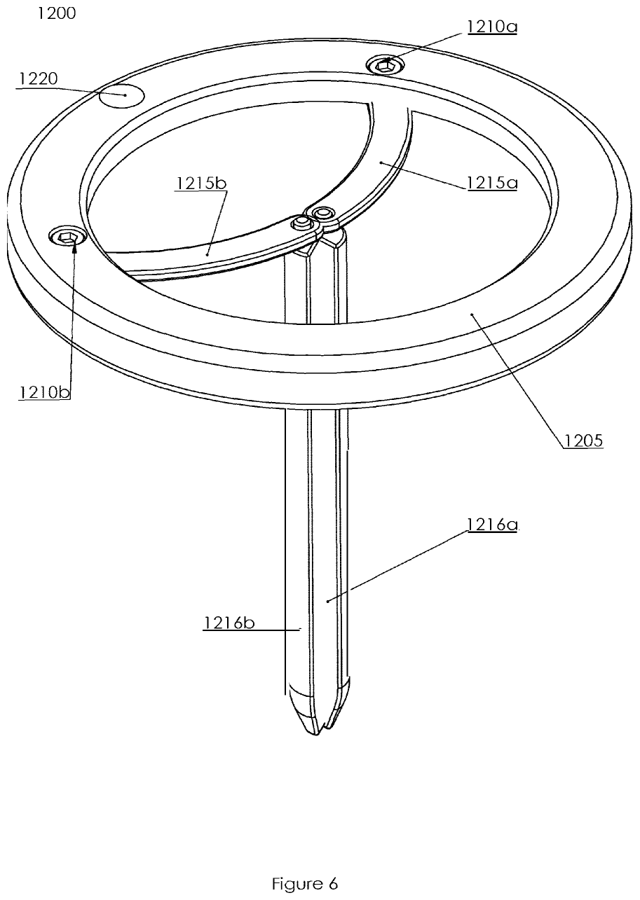

Referring now to FIG. 6, a ring retractor 1200 is shown. Ring retractor 1200 comprises a rigid ring 1205 having a coupling aperture 1220 extending therethrough and at least two blade assemblies extending from the distal face thereof, with each blade assembly comprising a threaded pivot bolt 1210 (a and b) coupled to the rigid ring 1205 (a and b) by means of a complementary threaded hole in the ring, an arm 1215 (a and b) pivotably coupled about the pivot bolt 1210 (a and b), with the arm 1215 (a and b) extending from the periphery of rigid ring 1205 to the center thereof and having a retractor blade 1216 (a and b) pivotably coupled to the arm 1215 (a and b) and extending distally therefrom.

Aperture 1220 is sized and shaped to complimentarily receive the post 1122 therewithin.

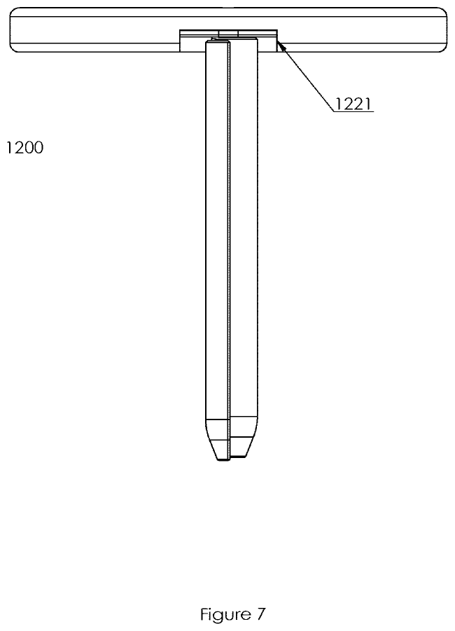

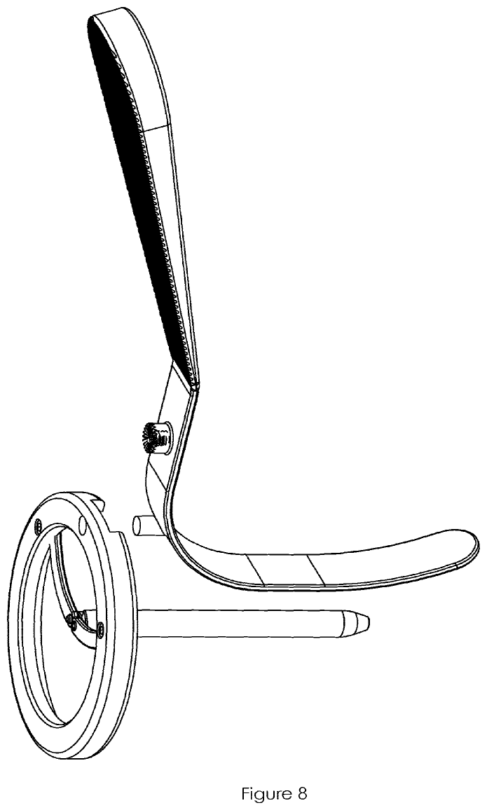

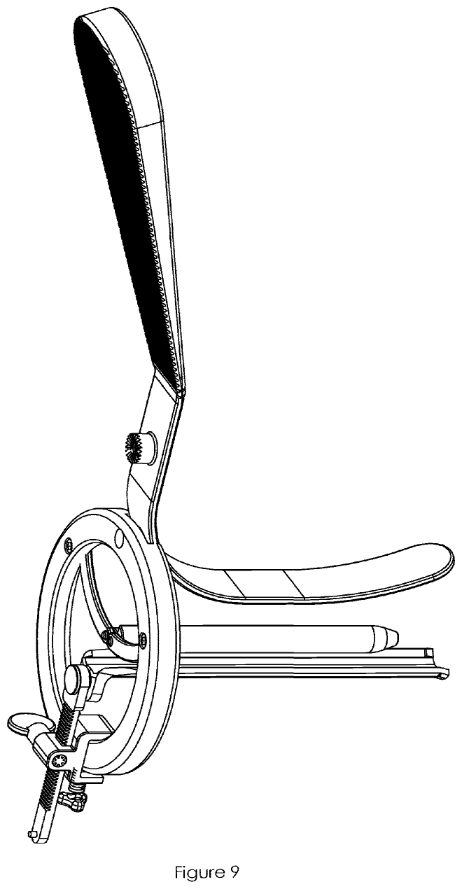

Referring now to FIG. 7, a distal cutout 1221 disposed upon the distal face of rigid ring 1205 is shown, where distal cutout 1221 is a portion of material removed from the distal face of the rigid ring 1205 sized and shaped to firmly accommodate coupling profile 1126 therewithin with minimal lateral movement.

A method of assembling and using the present invention will now be described. Initially, a handheld retractor 1100 is provided and grasped about its grip by a surgeon or operator. The blade 1120 is placed into in incision disposed upon a patient's body and the handle of the retractor translated in the general direction of the axis defined by the length of the handle, thereby pulling back tissue layers and partially exposing an operative site. At this stage, the handheld retractor may be mounted to a table arm (not shown) to be held in place about ribbed post 1123 or a table arm clamp engaging about the body of the handheld retractor. Next, the ring retractor 1200 is provided in a “closed” configuration, with the distance between its blades reduced to a minimum. The blades are inserted into the space created by the blade of the handheld retractor and placed atop the handheld retractor such that post 1122 is inserted into aperture 1220 and the opposing vertical sides of distal cutout 1221 abut the sides of coupling profile 1126 such that the ring retractor 1200 is firmly seated upon handheld retractor 1100. According to certain embodiments of the present disclosure, a clamping feature adds rigidity to the coupling of the handheld retractor 1100 to the ring retractor 1200.

Next, the opposing blades 1216 (a and b) of the ring retractor 1200 are manually articulated by an operator into an open configuration wherein a gap is created between them. Once a satisfactory gap has been created, the position of the blades 1216 (a and b) is secured by tightening the bolts 1210 (a and b) against the ring retractor 1200.

Next, the muscle retractor assembly 1300 is inserted into the operative site with the blade 1310 extending into the newly created operative space with the clamp 1330 secured about the body of ring retractor 1200. Finally, the stepped dilators are inserted into the operative space, including for instance the psoas muscle initially about a guidewire placed by the operator, first with dilator 1410 traveling over the guidewire and dilating and first area and finally with the dilator 1420 traveling over dilator 1410 creating the assembly approximated by FIGS. 10A and 10B.

Although the present invention has been described in the section above with reference to specific shapes and structures, one having ordinary skill in the mechanical arts will appreciate that the foregoing is one exemplary implementation of the claims which are appended below.

What is claimed is:

1. A retractor system comprising:
a handheld retractor comprising:
a blade disposed at a distal end;
a handle disposed at a proximal end; and
a coupling profile between the blade and the hand-grip; and
a ring retractor coupled to the handheld retractor comprising:
an armature defining a rigid ring;
a first blade assembly coupled to the armature by a first pivot bolt; and
a second blade assembly coupled to the armature by a second pivot bolt.

2. The retractor system of claim 1, wherein the armature further comprises a distal cutout having opposing vertical sides such that each of the opposing vertical sides abut a side of the coupling profile of the handheld retractor.

3. The retractor system of claim 2, further comprising:
a first engagement feature disposed between the blade and the handle of the handheld retractor; and
a second engagement feature disposed on the armature of the ring retractor,
wherein the first engagement feature and second engagement feature are complementary structures configured for rigidly coupling the retractor atop the handheld retractor.

4. The retractor system of claim 3, wherein the first engagement feature is a post and the second engagement feature is an aperture disposed within the distal cutout of the armature.

5. The retractor system of claim 1, further comprising a ribbed post having an interior thread and a plurality of teeth on a face normal to the thread.

6. The retractor system of claim 1, wherein the first blade assembly comprises:
a first arm pivotably coupled to the armature by the first pivot bolt; and
a first retractor blade pivotably coupled to the first arm and extending distally therefrom.

7. The retractor system of claim 6, wherein the second blade assembly comprises:
a second arm pivotably coupled to the armature by the second pivot bolt; and
a second retractor blade pivotably coupled to the second arm and extending distally therefrom.

8. The retractor system of claim 6, wherein the first arm is pivotably coupled to the armature by the first pivot bolt at a distal face of the armature and extends inwardly from a periphery of the armature.

9. The retractor system of claim 8, wherein the second arm is pivotably coupled to the armature by the second pivot bolt at the distal face of the armature and extends inwardly from the periphery of the armature.

10. The retractor system of claim 1, further comprising a muscle retractor coupled to the armature, wherein the muscle retractor comprises a muscle retractor blade linearly positionable relative to the armature.

11. The retractor system of claim 10, wherein the muscle retractor comprises:
a clamp having a body secured to the armature;
a linear gear extending inwardly from a periphery of the armature through the body of the clamp; and
the muscle retractor blade pivotably coupled to the linear gear and extending distally therefrom.

12. The retractor system of claim 11, wherein the linear gear further comprises:
a pivot at a distal end of the linear gear positioned within the periphery of the armature;
an upwardly extending pin at a proximal end of the linear gear; and
a plurality of teeth defining a gear train disposed between the distal and proximal ends of the linear gear.

13. The retractor system of claim 11, wherein the muscle retractor blade further comprises an elongated body having a curved cross-section with a pair of opposing faces turned inwards towards one another and a connecting surface disposed between the pair of opposing faces.

14. The retractor system of claim 13, wherein the muscle retractor blade further comprises a ridge positioned between each of the opposing faces in the pair of opposing faces and the connecting surface, thereby defining a rail.

15. The retractor system of claim 1, further comprising a plurality of stepped dilators.

16. The retractor system of claim 1, wherein the handheld retractor is a Deaver retractor.

17. The retractor system of claim 1, wherein the blade of the handheld retractor is an arcuate blade.
